(19) United States
(12) Patent Application Publication
(10) Pub. No.: US 2016/0184899 A1
(43) Pub. Date: Jun. 30, 2016

(54) METHOD FOR PREPARING A DENDRIMER TYPE OR DENDRIMER-DERIVED METAL NANOSTRUCTURE IN LIQUID-LIQUID INTERFACE AND DENDRIMER TYPE OR DENDRIMER-DERIVED METAL NANOSTRUCTURE PREPARED BY SAME

(71) Applicant: INDUSTRY-UNIVERSITY COOPERATION FOUNDATION SOGANG UNIVERSITY, Seoul (KR)

(72) Inventors: Taewook Kang, Seoul (KR); Sunil Jeong, Seoul (KR); Chi Won Lee, Seoul (KR); Yong Hee Shin, Gyeongsangnam-do (KR)

(73) Assignee: Industry-University Cooperation Foundation Sogang University, Seoul (KR)

(21) Appl. No.: 14/906,851

(22) PCT Filed: Nov. 20, 2013
(86) PCT No.: PCT/KR2013/010568

§ 371 Date: Jan. 21, 2016

(30) Foreign Application Priority Data
Jul. 23, 2013 (KR) 10-2013-0087023

(51) Int. Cl. B22F 9/24; B22F 1/00

(52) U.S. Cl. CPC B22F 9/24; B22F 1/0018; B22F 2009/245; B22F 2001/0037; B22F 2301/255; B22F 2304/054; B22F 2998/10; B82Y 5/00

(57) ABSTRACT

A dendrimer type or dendrimer-derived metal nanostructure may be very easily obtained from a metal precursor and a reducing agent in a liquid-liquid interface between different liquids which form the interface. The metal nanostructure may have, particularly, a low-dimensional structure. In addition, a plurality of nanogaps may be formed between many small branches.

### TECHNICAL FIELD

The present invention relates to a method for preparing a dendrimer type or dendrimer-derived metal nanostructure in a liquid-liquid interface and a dendrimer type or dendrimer-derived metal nanostructure prepared thereby. The technology disclosed in the present disclosure can be useful in wide variety of fields such as environmental, biological, energy and medical applications including molecular detection, catalyst, drug delivery, biomedical applications such as tailored therapy in cellular or molecular level using photothermal effect, application to meta materials for manufacturing of invisibility cloak, etc., and solar concentrator, etc.

### BACKGROUND ART

Researches have been conducted on formation of nanometer-sized nanogaps on a metal nanoparticle. It is a promising technology of fine-tuning the nanoparticle structure which allows generation of an electromagnetic field around the particle by concentrating incident light from outside.

Representative examples of the existing nanogap forming technologies may include ensemble nanostructures (dumbbell type, core-shell type, etc.) connected by biomolecules such as DNA, asymmetric nanostructures utilizing the steric hindrance effect (semishells) and complex nanostructures using the galvanic corrosion effect.

However, it is the inventors' observation that for the existing metal nanostructures, only limited number of nanogaps can be formed per single metal nanoparticle and, therefore, there is a limitation in achieving enhanced localized electromagnetic field over a large area of a single metal nanoparticle.

As a specific example, a single nanoparticle having a nanogap formed between a core material and a shell material and a method for preparing the same are known (WO 2012/070893).

However, it is the inventors' observation that this technology requires a complicated process of linking the core material with the shell material using a linker material such as DNA to form the nanogap and the formation of the nanogap is also limited.

As well, synthesis of a gold nanorod dimmer forming 5 nm-sized nanogaps using an on-wire lithography process has been reported (Dispersible Gold Nanorod Dimer with Sub-5 nm Gaps Local Amplifiers for Surface-Enhanced Raman Scattering, Nano Letters, Chad A. Mirkin et al. 2012, 2828-3832).

However, it is the inventors' observation that this technology is also limited in the location where the nanogaps are formed and to thus the area of enhanced electromagnetic field is also limited.

### SUMMARY OF THE INVENTION

The embodiments of the present invention are directed to providing a method for extremely easily preparing a dendrimer type or dendrimer-derived metal nanostructure and a dendrimer type or dendrimer-derived metal nanostructure having, particularly, a low-dimensional structure.

Specifically, the embodiments of the present invention are directed to providing a method for preparing a dendrimer type (branched type) metal nanostructure having subbranches or a dendrimer-derived metal nanostructure that has grown from the dendrimer very conveniently and easily.

The embodiments of the present invention are also directed to providing a dendrimer type or dendrimer-derived metal nanostructure having, in particular, a low-dimensional structure. By providing such low-dimensional dendrimer type (branched type) metal nanostructure with subbranches, a plurality of nanogaps can be formed easily at various locations and increased surface area per given volume and enhanced localized electromagnetic field over a large area of a single metal nanostructure can be achieved. In addition, the dendrimer type or dendrimer-derived metal nanostructure may have useful properties of, for example, providing a path through which a detected molecule and a drug can move freely, activating optical properties in the biologically transparent near-infrared range, etc.

In the embodiments of the present invention, provided is a method for preparing a dendrimer type or dendrimer-derived metal nanostructure, including obtaining a dendrimer type or dendrimer-derived metal nanostructure from a metal precursor and a reducing agent capable of reducing the metal precursor at a liquid-liquid interface between liquids which are different with each other and form the interface.

In an exemplary embodiment, the preparation method may include: locating a metal precursor and a reducing agent capable of reducing the metal precursor at a liquid-liquid interface between different liquids which are different with each other and form the interface; and gathering a dendrimer type or dendrimer-derived metal nanostructure from the interface.

In an exemplary embodiment, the preparation method may include: inhibiting the reduction of the metal precursor in the liquid other than the interface.

In an exemplary embodiment, in the preparation method, a plurality of branches may grow anisotropically from a metal nanoparticle nucleus along horizontal and vertical directions at the interface. Herein, a primary branch may grow from the metal nanoparticle nucleus and n-th (n is an integer which is 2 or more) branches may grow from the primary branch. The resulting metal nanostructure may be, as will be described below, a low-dimensional dendrimer type metal nanostructure having nanogaps between a plurality of branches or a dendrimer-derived metal nanostructure wherein the branches have further grown from the dendrimer type metal nanostructure. In an exemplary embodiment, the dendrimer type metal nanostructure may have a 2-dimensional or 1-dimensional structure wherein a plurality of branches are formed and nanogaps are present between the branches. Details about the dendrimer type metal nanostructure will be described below.

In an exemplary embodiment, in the preparation method, the interface may be provided as one of the liquids forms a droplet in another liquid.

In an exemplary embodiment, the preparation method may include: forming an interface by providing a first liquid (e.g., water) and a second liquid (e.g., an oil); and providing a metal precursor and a reducing agent to the interface.

In an exemplary embodiment, the preparation method may include: dissolving a metal precursor and a reducing agent in a first liquid (e.g., water); and forming an interface by providing a second liquid (e.g., an oil) to the first liquid in which the metal precursor and the reducing agent are dissolved.

The first liquid may contain water or may be water, and the second liquid may contain an oil or may be an oil. Herein, the oil may be, for example, phospholipid-based oil. More specifically, it may be, for example, olive oil, oleic acid or linoleic acid.

As described above, in an exemplary embodiment, the interface may be provided by forming a droplet. That is, when water and an oil are used, the interface may be provided as the oil forms a droplet in the water. Alternatively, the interface may be provided as the water forms a droplet in the oil.

In an exemplary embodiment, the pH of the water in which the metal precursor and the reducing agent are dissolved may be controlled to 3-4.

In an exemplary embodiment, the preparation method may be conducted at or above the melting point of the oil and at or below 30° C. For example, when oleic acid is used, it may be conducted at or above 16° C. and at or below 30° C.

In an exemplary embodiment, a metal of the metal nanostructure may be a transition metal. For example, the metal of the metal nanostructure may be one or more metal selected from a group consisting of Ag, Au, Cu, Pt, Fe, Co, Ni, Ru, Rh and Pd. And, the metal of the metal precursor may be specifically Au.

In an exemplary embodiment, HAuCl₄.3H₂O may be used as the metal precursor and hydroxylamine hydrochloride (NH₂OH.HCl) may be used as the reducing agent.

As well, in the embodiments of the present invention, provided is a dendrimer type or dendrimer-derived metal nanostructure, wherein the metal nanostructure has a 2-dimensional structure or a 1-dimensional structure.

In an exemplary embodiment, the metal nanostructure may be a dendrimer type metal nanostructure and the metal nanostructure may have a 2-dimensional or 1-dimensional structure wherein a plurality of branches are formed and nanogaps are present between the branches.

In an exemplary embodiment, the metal nanostructure may have a 2-dimensional structure with horizontal and vertical sizes of 10 nm or more and 100 nm or less and a thickness of 1-10 nm, or a 1-dimensional structure with one of horizontal and vertical sizes of 10 nm or more and 100 nm or less and the other vertical or horizontal size and a thickness of 1-10 nm, respectively.

In an exemplary embodiment, the metal nanostructure may have a primary (first) branch that has grown from a metal nanoparticle nucleus and n-th (n is an integer which is 2 or more) branches that have grown from the primary branch, and nanogaps may be present between the primary branch and the n-th branch and/or between the n-th branches.

For example, if n is 2, the metal nanostructure may have a primary branch that has grown from a metal nanoparticle nucleus and secondary branches that have grown from the primary branch, and nanogaps may be present between the primary branches and the secondary branches.

And, for example, if n is 3 or more, the metal nanostructure may have secondary branches that have grown from the primary branch and may further have n-th (n is an integer which is 3 or more) branches that have grown from the secondary branches.

That is, the n-th branch may refer to, for example, a secondary branch (n=2) that has grown from the primary branch, a tertiary branch (n=3) that has grown from the secondary branch, a quaternary branch (n=4) that has grown from the tertiary branch, a quinary branch (n=5) that has grown from the quaternary branch, . . . a n-th branch that has grown from a (n−1)-th branch (that has grown from a (n−2)-th branch). A plurality of nanogaps may be formed between the primary branch and the n-th branch and/or between the n-th branches.

In an exemplary embodiment, the number of branches of the n-th branch (n=1 or more) may be two or more for each n-th branch.

In an exemplary embodiment, preferably, the metal nanostructure may have horizontal and vertical sizes of 50-60 nm, respectively and a thickness of 4-5 nm.

In an exemplary embodiment, the size of the nanogap may be 10 nm or less and equal to or more than the inter-lattice distance of the metal atom. Specifically, it may be 1-10 nm or 2-8 nm.

In an exemplary embodiment, the surface area of the metal nanostructure may be 2-3 times or 2.5-3 times as compared to that of a spherical particle of the same volume.

In an exemplary embodiment, a metal of the metal nanostructure may be a transition metal. For example, it may be one or more selected from a group consisting of Ag, Au, Cu, Pt, Fe, Co, Ni, Ru, Rh and Pd, specifically Au.

According to the embodiments of the present invention, a dendrimer type or dendrimer-derived metal nanostructure may be prepared very easily in a liquid-liquid interface. As well, a low-dimensional dendrimer type or dendrimer-derived metal nanostructure may be provided as contrary to the existing technology. Accordingly, small branches may be formed in the low-dimensional structure and the metal nanostructure may have a high surface-area-to-volume ratio due to the small branches. Also, nanogaps present between the small branches of the low-dimensional dendrimer type metal nanostructure may provide a strong electromagnetic field over a wide area. In addition, there are also useful properties that a detected molecule or a drug may move freely around the low-dimensional dendrimer type or dendrimer-derived metal nanostructure, and optical properties may be activated in a biologically transparent near-infrared range, etc.

The dendrimer type or dendrimer-derived metal nanostructure according to the embodiments of the present invention may be useful in wide variety of environmental, biological, energy and medical applications, etc. including molecular detection, catalyst, drug delivery, biomedical applications such as tailored therapy in cellular or molecular level using photothermal effect, application to metamaterials for manufacturing of, e.g., an invisibility cloak, and solar concentrator, etc.

### BRIEF DESCRIPTION OF THE DRAWINGS

FIG. 1 schematically illustrates a preparation method according to an exemplary embodiment of the present invention.

FIG. 2 shows a photographic image and schematics illustrating formation of a dendrimer type metal (e.g., gold) nanostructure in a droplet liquid-liquid interface according to an exemplary embodiment of the present invention.

FIG. 3 shows a computer simulation result showing the electromagnetic field effect of a dendrimer type metal nanostructure according to an exemplary embodiment of the present invention.

FIGS. 4 and 5 show images of a dendrimer type metal nanostructure prepared in Example 1. FIG. 4 is a TEM image and FIG. 5 is an AFM image.

FIG. 6 shows a TEM image of a dendrimer type metal nanostructure obtained in Example 2 (droplet liquid-liquid interface).

FIG. 7 shows activation of Raman signals by a dendrimer type metal nanostructure obtained in Example 2.

FIGS. 8 and 9 show images of a metal nanostructure which is further grown from a dendrimer type metal nanostructure in Example 3. FIG. 8 is a TEM image and FIG. 9 is an AFM image.

FIGS. 10 and 11 show images of a dendrimer-derived metal nanostructure obtained in Example 4. FIG. 10 is a TEM image and FIG. 11 is an AFM image.

### DETAILED DESCRIPTION

In the present disclosure, in the term metal nanoparticle or metal nanostructure, ‘nano’ means that the size of the nanoparticle or the nanostructure (horizontal size, vertical size, thickness, particle diameter, etc.) is smaller than 1 micrometer, i.e., 1000 nm. But, a ‘nanogap’ means a gap of 10 nm or less in size.

In the present disclosure, a ‘dendrimer’ means a branched structure. Not only a structure having a plurality of branches, one having branches only on the peripheral portion due to a continuing growth of the branches is also included.

In the present disclosure, ‘dendrimer-derived’ means a structure derived from a dendrimer type structure. Although it may be difficult to be called as a dendrimer type because branches are hardly observed due to the continuing growth of the branches of the dendrimer type structure, it may be referred as a structure derived from a dendrimer type metal nanostructure. Accordingly, it is to be understood that a metal nanostructure derived from a dendrimer type metal nanostructure of the present disclosure which is no more a dendrimer type due to the continuing growth of branches may be included in the scope of the present disclosure.

In the present disclosure, a liquid-liquid interface may include, not only the precise interface itself in a strict sense, but also the surroundings of the interface.

In the present disclosure, a metal nanoparticle nucleus may refer to a particle made from a reduction of a metal nanoprecursor before the growth of branches.

In the present disclosure, low-dimensional means a dimension which is lower than 3 dimensions. That is, it means 2-dimensional or 1-dimensional.

In the present disclosure, 3-dimensional means that the horizontal size, vertical size and thickness of a structure does not have one or more order of magnitude difference. That is, if the horizontal size, vertical size and thickness are similar in size to the extent that they are not different from each other by one or more order of magnitude, the structure may be called a 3-dimensional structure.

In the present disclosure, 2-dimensional means that, although the horizontal size and vertical size of a structure does not have at least one order of magnitude difference, the horizontal size and a thickness and the vertical size and the thickness have at least one order of magnitude difference. That is, although the horizontal size and the vertical size are similar in size to the extent that they are not different from each other by one or more order of magnitude, if the thickness is different from the horizontal size and the vertical size by one or more order of magnitude, the structure may be called a 2-dimensional structure (e.g., a plate-shaped structure).

In the present disclosure, 1-dimensional means that the horizontal size and the vertical size of a structure have at least one order of magnitude difference and the horizontal size and a thickness or the vertical size and the thickness have at least one order of magnitude difference. For example, if a structure is long along the horizontal (or vertical) direction and the vertical (or horizontal) size is different from the horizontal (or vertical) size by at least one order of magnitude and the thickness is also different from the horizontal (or vertical) size by at least one order of magnitude, it may be called a 1-dimensional structure (e.g., a rod-shaped structure).

For reference, the expression that A and B are different by at least one order of magnitude is frequently used expression meaning that the sizes of A and B are different by at least 10 times.

In embodiments of the present invention, a dendrimer type or dendrimer-derived metal nanostructure may be obtained from a metal precursor and a reducing agent capable of reducing the metal precursor at a liquid-liquid interface between liquids which are different with each other and form the interface.

At the liquid-liquid interface, a particle nucleus is formed from oxidation-reduction reaction of the metal precursor and the reducing agent and branches are formed from the nucleus through specific growth due to a surface diffusion-controlled reaction mechanism. The branches of the metal nanostructure grow anisotropically along the horizontal or vertical direction. The manufactured metal nanostructure may be a low-dimensional dendrimer structure, e.g., a plate-shaped or a rod-shaped structure, having many small branches or may be a dendrimer-derived structure.

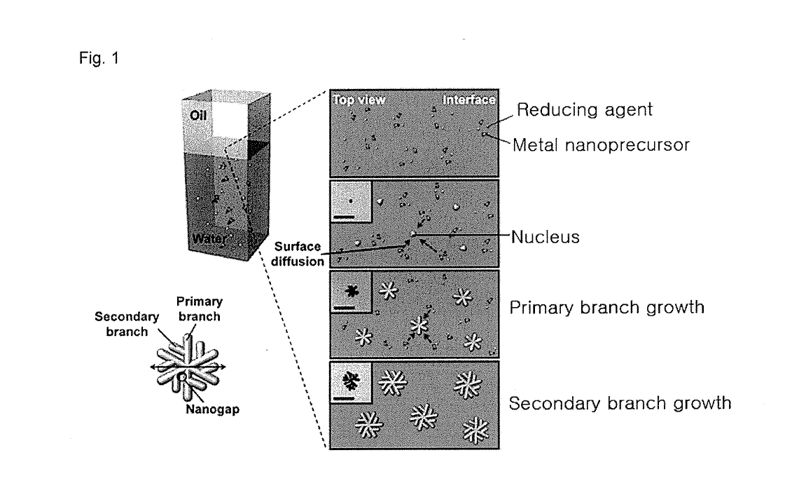

FIG. 1 schematically illustrates a preparation method according to an exemplary embodiment of the present invention. Although the shape of the metal nanostructure is schematically shown in FIG. 1, the shape as shown in FIG. 1 is only exemplary and it is to be understood that the shape or method of the metal nanostructure is not particularly limited to those shown in FIG. 1.

Referring to FIG. 1, in an exemplary embodiment of the present invention, a dendrimer type or dendrimer-derived metal nanostructure having a plurality of branches may be prepared easily at a liquid (exemplified by water in FIG. 1)—liquid (exemplified by an oil in FIG. 1) interface according to a surface diffusion-controlled reaction occurring at the liquid-liquid interface. This preparation method is advantageous in terms of preparation yield and process efficiency because the dendrimer type or dendrimer-derived metal nanostructure, particularly a low-dimensional metal nanostructure, may be prepared simply using commonly used immiscible liquids such as water and oil.

Specifically, when a metal precursor and a reducing agent capable of reducing the same are present at a liquid-liquid interface of immiscible different liquids which form the interface, a particle nucleus is formed from oxidation-reduction reaction of the metal precursor and the reducing agent and branch growth occurs from the nucleus as the diffusion rate of metal atoms around the nucleus is controlled.

That is, in a metal nanoparticle growing along the liquid-liquid interface, lateral growth is predominant due to the difference in diffusion rate in the liquids and the interface (the surface diffusion rate of the metal precursor is very slow at the interface than at the liquids) and a low-dimensional structure may be formed as a plurality of branches grow anisotropically along the horizontal and vertical directions of the particle (see FIG. 1). As will be described later, a plurality of nanogaps are formed between these branches.

If the branches in the dendrimer type metal nanostructure grow further, a structure wherein the branches are present only on the peripheral portion of the particle (e.g., a sea urchin-shaped structure) may be obtained. And, if necessary, the branches may be grown further such that the branches present on the peripheral portion nearly disappear (e.g., a plate-shaped structure with a constant thickness). Such a structure cannot be seen as a dendrimer type metal nanostructure because it has few branches, but it is called a dendrimer-derived metal nanostructure because it is derived from a dendrimer.

When the dendrimer type metal nanostructure or dendrimer-derived metal nanostructure having many small branches is formed, it may be gathered to obtain the metal nanoparticle. For example, the solution near the interface may be gathered and the dendrimer type or dendrimer-derived nanostructure may be obtained through a post-treatment process such as centrifugation.

Meanwhile, it may be necessary to inhibit the reduction of the metal precursor in the liquid phase other than the interface so that the amount of formed nuclei in the liquid phase other than the interface through reduction of the metal precursor to the metal nanostructure is less than that of formed at the interface. As will be described below, in an exemplary embodiment, the pH of the solution in which the metal precursor and the reducing agent are dissolved may be controlled, for example, to 3-4, so that the reduction of the metal precursor in the liquid phase other than the interface is inhibited and the reduction occurs predominantly at the interface.

In an exemplary embodiment, the preparation may be conducted at or above the melting point of the oil (i.e., at a temperature where the oil is not solidified) and at or below 30° C. For example, when oleic acid is used, it may be conducted at 16-30° C. Because the melting point of oleic acid is 16° C., if the preparation is conducted at or below 15° C., it may be difficult to form a liquid-liquid interface because the oil is solidified. And, if the temperature is higher than 30° C., the diffusion-controlled mechanism of dendrimer type metal nanostructure formation at the interface may not be operable due to a too fast rate of diffusion of the metal precursor or the reducing agent.

In an exemplary embodiment, two liquids, i.e., a first liquid and a second liquid, which are immiscible with each other and form an interface may be used. The first liquid may be a water-based liquid including water. Also, when the first liquid may be a water-based liquid including water and the second liquid may include an oil. As a non-limiting example, the first liquid may be water and the second liquid may be an oil. For example, the oil may be olive oil, oleic acid, linoleic acid, etc.

As a non-limiting example, the interface may be formed by providing a metal precursor and a reducing agent to the first liquid (e.g., water) and then providing a second liquid (e.g., an oil) to the first liquid (e.g., water) in which the metal precursor and the reducing agent are dissolved. The metal precursor and the reducing agent are to be dissolved in the first liquid or the second liquid. For example, when water and an oil are used, the metal precursor and the reducing agent are dissolved in water.

As a non-limiting example, after forming the interface of the first liquid and the second liquid, the metal precursor and the reducing agent may be provided to the interface. For this, the metal precursor and the reducing agent may be injected (provided) to the interface using, for example, a syringe.

In another exemplary embodiment, the interface may be formed as one of the different liquids forms a droplet in another liquid.

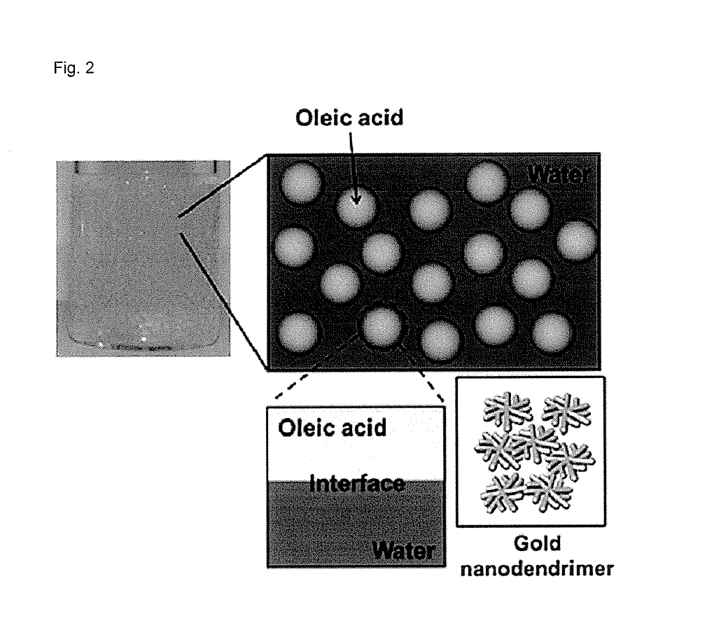

FIG. 2 shows a photographic image and schematics illustrating formation of a dendrimer type metal (e.g., gold) nanostructure in a droplet liquid-liquid interface according to an exemplary embodiment of the present invention.

As shown in FIG. 2, a plurality of droplets may be formed by quickly injecting a second liquid (e.g., oleic acid) while stirring a first liquid (e.g., water) to which the metal precursor and the reducing agent have been provided.

Then, an interface is formed for each droplet, and a dendrimer type metal nanostructure or a dendrimer-derived metal nanostructure may be obtained at the interface through reduction of the metal precursor by the reducing agent, nucleus formation, anisotropic branch growth through surface diffusion-limited reaction, etc. as described above. As such, because the metal nanostructure may be obtained from the interface of each droplet, the dendrimer type or dendrimer-derived metal nanostructure may be obtained easily in large quantities with high yield.

Although formation of oil droplets by adding oil (e.g., oleic acid) to water is exemplified in FIG. 2, it is also possible to form water droplets in oil by reducing the amount of water and increasing the amount of the oil. When water droplets are formed in the oil as such, the metal precursor and the reducing agent remain dissolved in the droplet.

In an exemplary embodiment, a metal of the metal precursor may be a transition metal. For example, the metal of the metal precursor may be one or more selected from a group consisting of Ag, Au, Cu, Pt, Fe, Co, Ni, Ru, Rh and Pd, specifically Au.

As for a non-limiting example, the metal of the metal precursor may be gold (Au). For example, HAuCl₄.3H₂O may be used as the metal precursor and NH₂OH.HCl may be used as the reducing agent. The precursor and the reducing agent are added to water and pH is adjusted to 3-4. That is, if the precursor at an appropriate concentration (e.g., 1 mg/mL) is added to water, the pH of the water becomes 3-4. In this case, the reducing power of the reducing agent is decreased and the reduction in the liquid can be minimized or inhibited (prevented).

Then, oil is injected to the solution in which the metal precursor and the reducing agent are mixed in water to form a planar liquid/liquid interface or a droplet liquid-liquid interface. If it is desired to form the droplet liquid-liquid interface, the oil is injected quickly while stirring the mixture solution. The overall procedure is conducted at or above the solidification temperature of the oil and at or below 30° C. (i.e., approximately room temperature). Through this simple process, the dendrimer type or dendrimer-derived metal nanostructure may be obtained very easily as the reduction in the liquids is inhibited and the reduction in the interface is facilitated.

Hereinafter, the dendrimer type or dendrimer-derived metal nanostructure prepared by the preparation method according to an exemplary embodiment of the present disclosure is described in detail.

In an exemplary embodiment, the present disclosure may provide a nano-sized dendrimer type metal nanostructure, particularly a low-dimensional dendrimer type metal nanostructure, having a size of smaller than 1 μm, e.g., 300 nm or smaller or 200 nm or smaller, particularly 100 nm or smaller, having a plurality of branches and having nanogaps with a size of 10 nm or smaller present between the branches. The dendrimer type metal nanostructure having a plurality of branches is formed by the above-described preparation method and a plurality of nanogaps are formed between the branches.

Referring again to FIG. 1, in an exemplary embodiment, the metal nanostructure has a primary branch that has grown from the metal nanoparticle nucleus and secondary branches that have grown from the primary branch and has a plurality of nanogaps with a size of 10 nm or smaller present between the branches. Although only the secondary branches are shown in FIG. 1, tertiary branches may be further formed from the secondary branches and quaternary branches may be further formed from the tertiary branches. That is to say, the metal nanostructure obtained in an exemplary embodiment of the present disclosure may have n-th (n is an integer which is 2 or greater) branches that have grown from the primary branch. The n-th branch refers to a secondary branch (n=2) that has grown from the primary branch, a tertiary branch (n=3) that has grown from the secondary branch, a quaternary branch (n=4) that has grown from the tertiary branch, a quinary branch (n=5) that has grown from the quaternary branch, . . . , a n-th branch that has grown from a (n−1)-th branch (that has grown from a (n−2)-th branch). Also, in an exemplary embodiment, the number of branches of the n-th order except the primary branch may be two or more for each order.

In an exemplary embodiment, the present disclosure may provide a nano-sized dendrimer-derived metal nanostructure having a size of smaller than 1 μm, e.g., 300 nm or smaller or 200 nm or smaller, particularly 100 nm or smaller. In particular, the dendrimer-derived metal nanostructure may be a low-dimensional structure. When compared with the dendrimer type metal nanostructure having branches, the dendrimer-derived metal nanostructure may have a constant thickness in almost all portions.

The dendrimer-derived metal nanostructure may retain the characteristics of a plasmonic nanoparticle. Also, in particular, a low-dimensional structure may exhibit unique optical and electrical properties because free electrons are spatially confined. In addition, although the dendrimer-derived structure hardly has a branch structure, high reproducibility of optical signals can be expected in almost all locations of the structure because it can have a constant thickness in almost all portions. These characteristics may be usefully utilized in wide applications including manufacturing of functional devices, biomedical sensing and imaging, monitoring of catalytic reactions, etc.

In an exemplary embodiment, the present disclosure may provide a low-dimensional dendrimer type or dendrimer-derived metal nanostructure of 2 dimensions or 1 dimension. That is to say, the dendrimer type or dendrimer-derived metal nanostructure may be a 2-dimensional structure whose thickness is different from its horizontal size and vertical size by at least one order of magnitude or a 1-dimensional structure, for example, a structure which is long in the horizontal direction and its vertical size and thickness are different from its horizontal size by at least one order of magnitude.

In particular, in the low-dimensional dendrimer type metal nanostructure, the nanogaps present between the branches provide a large specific surface area (a larger surface area for the same volume) and provide a strong electromagnetic field over a large area.

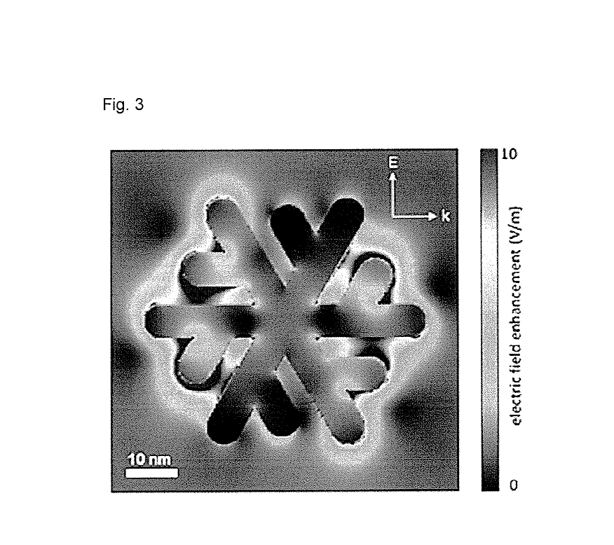

FIG. 3 shows a computer simulation result showing the electromagnetic field effect of a low-dimensional (2-dimensional) dendrimer type metal nanostructure according to an exemplary embodiment of the present disclosure.

As can be seen from the computer simulation result of FIG. 3, the nanogaps formed between the branches of the dendrimer type metal nanostructure provide a strong electromagnetic field enhancement effect. This effect is stronger as the number of higher-order subbranches (i.e., secondary or higher branches) is larger because more small-sized nanogaps can be formed.

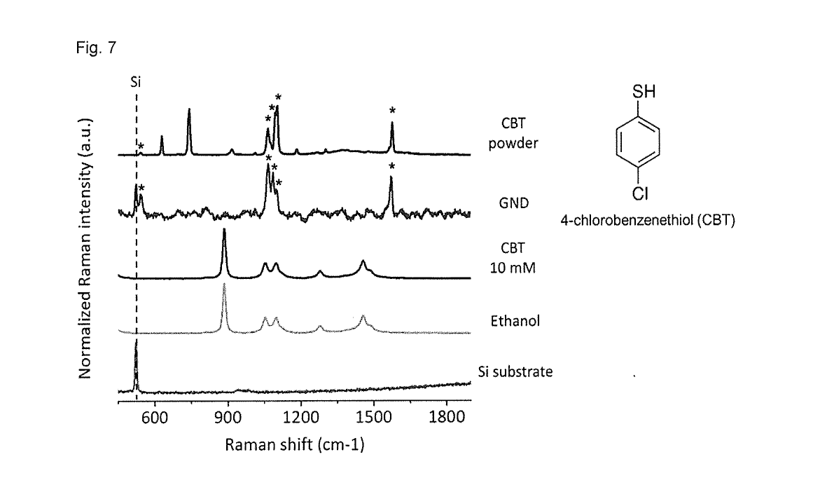

In addition, the low-dimensional dendrimer type metal nanostructure may have a significantly larger surface area as compared to a spherical particle of the same volume. In particular, optical properties may be further activated in the biologically transparent near-infrared range (see FIG. 7).

In an exemplary embodiment, the size of the nanogap may be 10 nm or smaller and equal to or larger than the inter-lattice distance of the metal atom. Specifically, it may be 1-10 nm or 2-8 nm.

In an exemplary embodiment, the horizontal and/or vertical size of the metal nanostructure (dendrimer type or dendrimer-derived metal nanostructure) may be, for example, 300 nm or smaller, 200 nm or smaller or 100 nm or smaller, more specifically 10-100 nm, 20-90 nm, 30-80 nm, 40-60 nm, 40-50 nm or 50-60 nm.

In an exemplary embodiment, the thickness of the metal nanostructure (dendrimer type or dendrimer-derived metal nanostructure) may be about 1-10 nm, 2-9 nm, 3-8 nm, 4-6 nm, 4-5 nm or 5-6 nm.

As a non-limiting example, the dendrimer type metal nanoparticle may be one having a horizontal size of about 50 nm and a vertical size of about 4 nm and having nanogaps with a size of 2-8 nm formed between subbranches (for reference, the horizontal size, vertical size and thickness may be measured by TEM and AFM as shown in FIGS. 3, 4, 5 and 8-11).

In an exemplary embodiment, the surface area of the metal nanostructure may be 2-3 times or 2.5-3 times that of a spherical particle of the same volume.

In an exemplary embodiment, the metal of the metal nanostructure may be a transition metal. For example, it may be one or more selected from a group consisting of Ag, Au, Cu, Pt, Fe, Co, Ni, Ru, Rh and Pd, specifically Au. In particular, a gold (Au) nanostructure will be useful in biomedical applications such as tailored therapy in cellular or molecular level.

The low-dimensional dendrimer type or dendrimer-derived metal nanostructure according to an exemplary embodiment of the present disclosure has unique structural and optical characteristics.

That is to say, the low-dimensional dendrimer type metal nanostructure obtained in an exemplary embodiment of the present disclosure has a high surface-area-to-volume ratio due to the low-dimensional subbranch structure. Also, the nanogaps present between the subbranches provide strong electromagnetic field over a large area. In addition, a detected molecule or a drug can move freely around the low-dimensional dendrimer type metal nanostructure and optical properties may be activated in the biologically transparent near-infrared range.

Due to these characteristics, the dendrimer type metal nanostructure can be used as a probe for detecting environmentally or biologically important molecules with high sensitivity or as a solar concentrator using the localized electromagnetic field formed between the plurality of nanogaps.

Also, the dendrimer-derived metal nanostructure may retain the characteristics of the plasmonic nanoparticle. In particular, a low-dimensional structure (e.g., a plate-shaped 2-dimensional structure) may exhibit unique optical and electrical properties because free electrons are spatially confined. In addition, high reproducibility of optical signals can be expected in almost all locations of the structure because it can have a constant thickness in almost all portions.

Because the dendrimer type or dendrimer-derived metal nanostructure has a low-dimensional structure at a size that can be directly applied to the human body, e.g., 100 nm or smaller, it can be useful in biomedical applications such as drug delivery or tailored therapy in cellular or molecular level using photothermal effect.

In addition, the structure having a low-dimensional structure of 2 dimensions or 1 dimension may also be useful in application to metamaterials (metallic materials much smaller in size than the wavelengths of the phenomena they influence) for manufacturing of, e.g., a militarily important invisibility cloak.

Hereinafter, the present disclosure will be described in detail through examples. However, the following examples are for illustrative purposes only and it will be apparent to those of ordinary skill in the art that the scope of the present disclosure is not limited by the examples.

Although oleic acid was used in the following examples, any other oil that can be obtained easily such as olive oil may also be used to prepare a low-dimensional dendrimer type or dendrimer-derived metal nanostructure.

In addition, although a gold nanoparticle precursor was used as a metal precursor in the following examples, other metal precursors may also be used to prepare a low-dimensional dendrimer type or dendrimer-derived metal nanostructure.

### EXAMPLE 1

In Example 1, a planar liquid/liquid interface was formed. After adding 12.8 mL of distilled water and 0.850 mL of a 1 mg/mL HAuCl₄.3H₂O solution to a 30-mL glass container, 37.5 μL of 0.003475 mg/mL NH₂OH.HCl was added as a reducing agent. After mixing homogenously, 2.8 mL of oleic acid was slowly introduced to the mixture solution such that an interface could be formed between the two liquids. After an interface was formed, 1 mL of the solution was gathered near the interface and a dendrimer nanostructure was obtained through centrifugation. All the procedure was conducted at 15° C. The obtained dendrimer type metal nanostructure can be resuspended in water or an organic solvent for use.

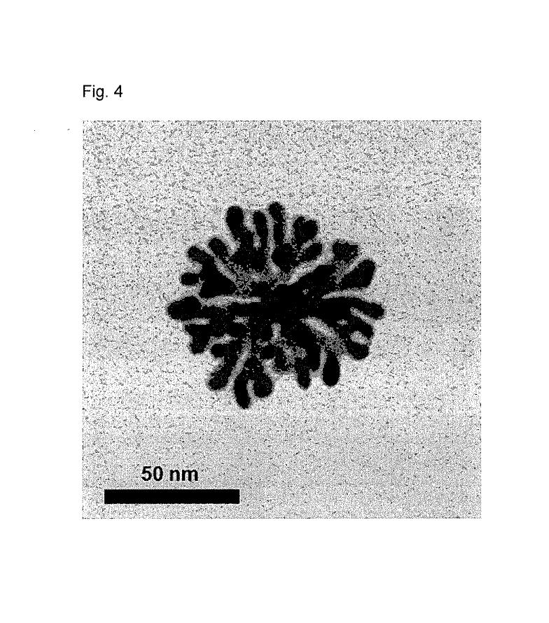

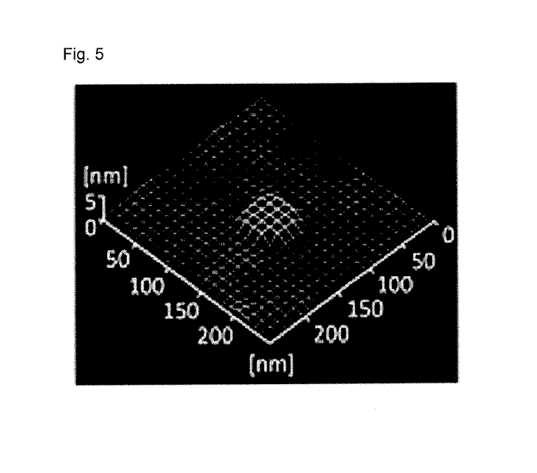

FIGS. 4 and 5 show images of the dendrimer type metal nanostructure prepared according to the present disclosure in Example 1. FIG. 4 is a TEM image and FIG. 5 is an AFM image.

### EXAMPLE 2

In Example 2, a droplet liquid-liquid interface was formed. After adding 13.225 mL of distilled water and 0.425 mL of a 1 mg/mL HAuCl₄.3H₂O solution to a 30-mL glass container, 37.5 μL of 0.003475 mg/mL NH₂OH.HCl was added as a reducing agent and the mixture was mixed homogenously. While the mixture solution was being stirred at a constant rate, 2.8 mL of oleic acid was quickly injected to the mixture solution to form a droplet liquid-liquid interface. After the formation of the droplet liquid-liquid interface was confirmed, stirring was stopped 10 minutes later. Within 30 seconds after the stirring was stopped, the mixture solution was separated into an aqueous solution and oleic acid in the form of droplets (see FIG. 2). Only the aqueous solution containing the dendrimer nanostructure was gathered and the dendrimer nanostructure was obtained through centrifugation. The dendrimer nanostructure can be resuspended in water or an organic solvent for use.

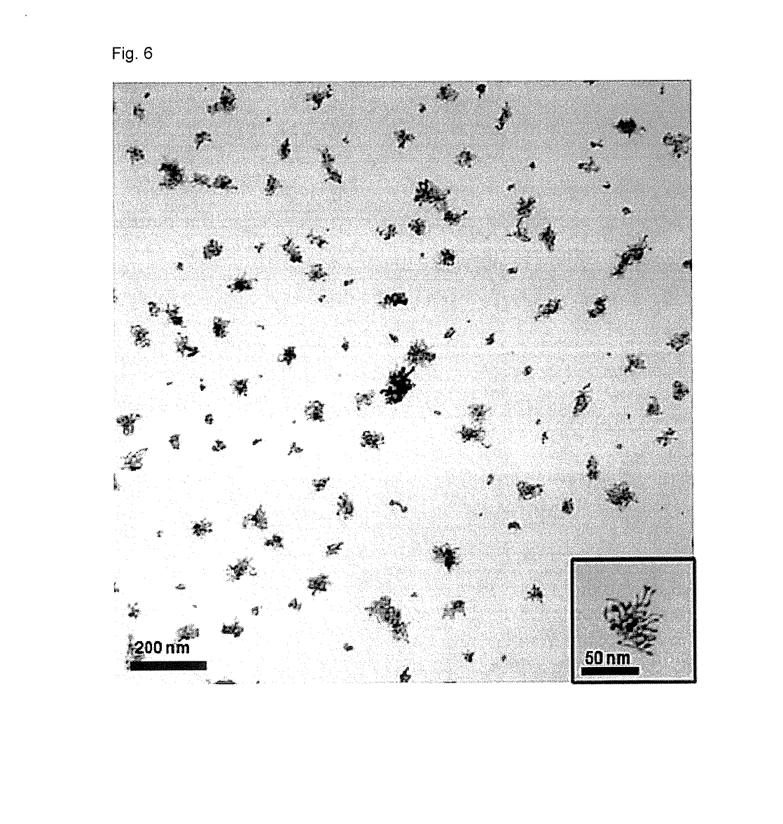

FIG. 6 shows a TEM image of the dendrimer type metal nanostructure obtained according to the present disclosure in Example 2 (droplet liquid-liquid interface).

Activation of optical properties was investigated using the dendrimer type metal nanostructure obtained in Example 2. FIG. 7 shows the activation of Raman signals by the metal nanostructure obtained according to the present disclosure in Example 2.

A molecule emits a Raman signal when it interacts with light. Because the Raman signal varies with the unique structure of the molecule, it can be usefully used to detect a particular molecule. The Raman signal is very strongly enhanced when there is a metal nanostructure around the molecule. FIG. 7 shows an example of detecting the chlorobenzenethiol (CBT) molecule by enhancing the Raman signal using a dendrimer type gold nanostructure according to an example embodiment of the present disclosure. The first (top) graph in FIG. 7 shows the characteristic Raman signal of chlorobenzenethiol. The third graph shows that the characteristic Raman signal is not observed when chlorobenzenethiol is present in a solution (ethanol) at low concentration. The second graph shows that the Raman signal (optical signal) of chlorobenzenethiol is enhanced by the dendrimer type gold nanostructure (GND; gold nanodendrimer) and appears again.

### EXAMPLE 3

A dendrimer type metal nanostructure was prepared in the same manner as in Example 1, except that branch growth was longer than in Example 1. In Example 1, the branch growth time was about 4 minutes after the formation of the interface. In Example 3, the branch growth time was about 1-2 minutes longer than in Example 1.

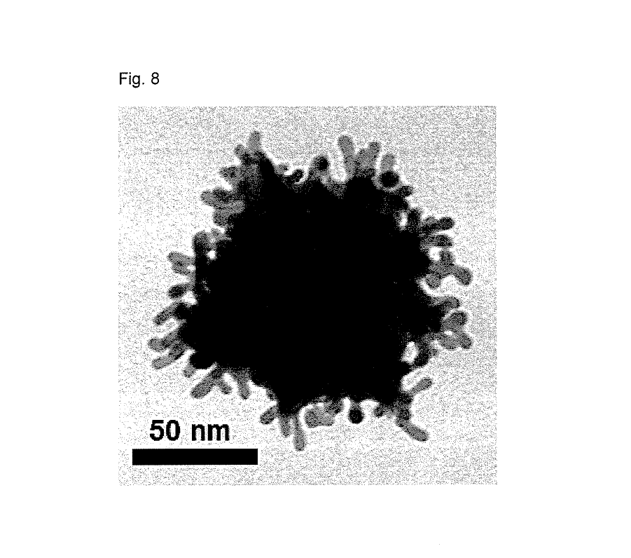

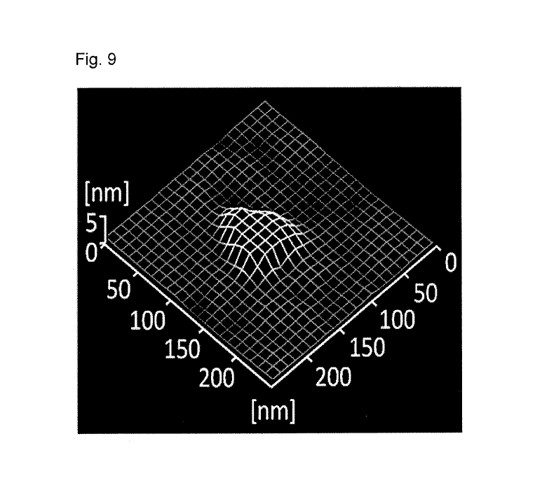

FIGS. 8 and 9 show images of the dendrimer type metal nanostructure grown further according to the present disclosure in Example 3. FIG. 8 is a TEM image and FIG. 9 is an AFM image.

It can be seen that a (sea urchin-shaped) gold nanostructure wherein the branches have grown further and remain only on the peripheral portion of the particle was obtained (see FIGS. 8 and 9). This structure is also a low-dimensional structure whose thickness is smaller by at least one order of magnitude than the horizontal and vertical lengths and has many nanogaps present in the peripheral portion. The horizontal and vertical lengths are 100-120 nm on average and the thickness is about 5 nm (see FIG. 9). This dendrimer type structure is also useful as the nanostructure obtained in Example 1.

### EXAMPLE 4

A dendrimer-derived metal nanostructure was prepared in the same manner as in Example 3, except that branch growth was about 2 minutes longer than in Example 3.

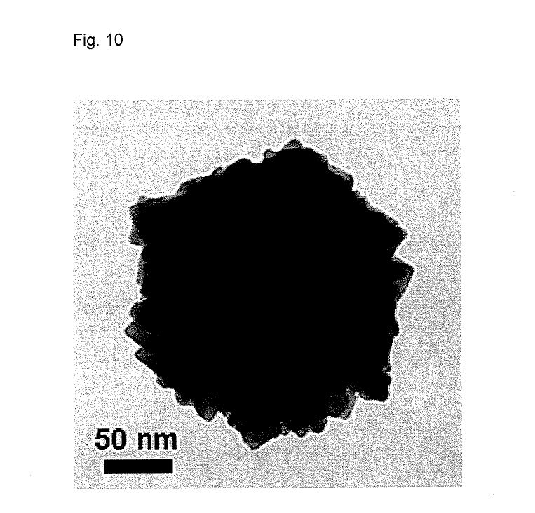

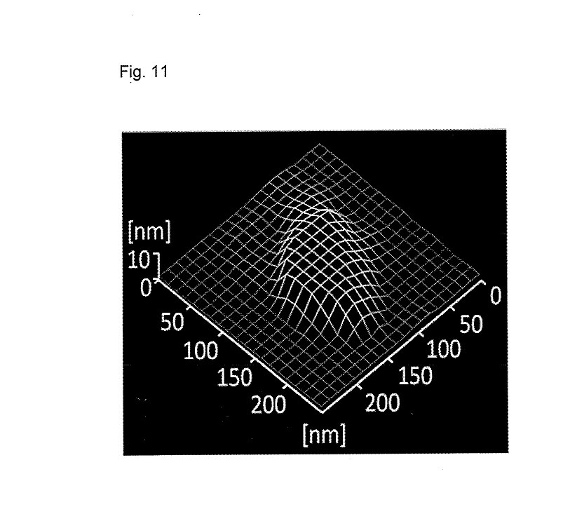

FIGS. 10 and 11 show images of the dendrimer-derived metal nanostructure obtained according to the present disclosure in Example 4. FIG. 10 is a TEM image and FIG. 11 is an AFM image.

The structure of Example 4 is one which has grown further from the sea urchin-shaped metal nanostructure obtained in Example 3. It can be seen that the branches that were present on the peripheral portion nearly disappeared and a plate-shaped structure with a nearly constant thickness was formed (see FIGS. 10 and 11). This structure is also a low-dimensional structure whose thickness is smaller by at least one order of magnitude than the horizontal and vertical lengths. The horizontal and vertical lengths are about 200 nm and the thickness is about 17 nm on average (see FIG. 11). It has a constant thickness in almost all portions as compared to the structures having branches. This structure may also be used in various applications such as biosensors or catalysts.

### INDUSTRIAL APPLICABILITY

The technology disclosed in the present disclosure may be useful in wide variety of environmental, biological, energy and medical applications including molecular detection, catalyst, drug delivery, biomedical applications such as tailored therapy in cellular or molecular level using photothermal effect, application to metamaterials for manufacturing of, e.g., an invisibility cloak, solar concentrator, etc.

1. A method for preparing a metal nanostructure, comprising obtaining a dendrimer type or dendrimer-derived metal nanostructure from a metal precursor and a reducing agent capable of reducing the metal precursor at a liquid-liquid interface between liquids which are different with each other and form the interface.

2. The method for preparing a metal nanostructure according to claim 1, which comprises: locating the metal precursor and the reducing agent capable of reducing the metal precursor at the liquid-liquid interface between liquids which are different with each other and form the interface; and gathering a dendrimer type or dendrimer-derived metal nanostructure from the interface.

3. The method for preparing a metal nanostructure according to claim 1, wherein the reduction of the metal precursor in the liquid other than the interface is inhibited.

4. The method for preparing a metal nanostructure according to claim 1, wherein a plurality of branches grow anisotropically from a metal nanoparticle nucleus along horizontal and vertical directions in the interface.

5. The method for preparing a metal nanostructure according to claim 4, wherein a primary branch grows from the metal nanoparticle nucleus and n-th (n is an integer which is 2 or more) branches grow from the primary branch.

6. The method for preparing a metal nanostructure according to claim 1, wherein the dendrimer type metal nanostructure has a 2-dimensional or 1-dimensional structure wherein a plurality of branches are formed and nanogaps are present between the branches, and the dendrimer-derived metal nanostructure has a 2-dimensional or 1-dimensional structure.

7. The method for preparing a metal nanostructure according to claim 1, wherein the interface is provided as one of the different liquids forms a droplet in another liquid.

8. The method for preparing a metal nanostructure according to claim 1, wherein a metal of the metal nanostructure is one or more metal selected from a group consisting of Ag, Au, Cu, Pt, Fe, Co, Ni, Ru, Rh and Pd.

9. The method for preparing a metal nanostructure according to claim 1, comprising: forming the interface by providing water and oil; and providing the metal precursor and the reducing agent to the interface.

10. The method for preparing a metal nanostructure according to claim 1, comprising: dissolving the metal precursor and the reducing agent in water; and forming the interface by providing oil to the water in which the metal precursor and the reducing agent are dissolved.

11. The method for preparing a metal nanostructure according to claim 10, wherein the interface is provided as the oil forms a droplet in the water.

12. The method for preparing a metal nanostructure according to claim 10, wherein the interface is provided as the water forms a droplet in the oil.

13. The method for preparing a metal nanostructure according to claim 10, wherein the oil is olive oil, oleic acid or linoleic acid.

14. The method for preparing a metal nanostructure according to claim 10, wherein the pH of the water in which the metal precursor and the reducing agent are dissolved is controlled to 3-4.

15. The method for preparing a metal nanostructure according to claim 14, wherein the preparation is conducted at or above the melting point of the oil and at or below 30° C.

16. The method for preparing a metal nanostructure according to claim 15, wherein HAuCl₄.3H₂O is used as the metal precursor and hydroxylamine hydrochloride (NH₂OH.HCl) is used as the reducing agent.

17. A dendrimer type or dendrimer-derived metal nanostructure, wherein the metal nanostructure has a 2-dimensional structure or a 1-dimensional structure.

18. The metal nanostructure according to claim 17, wherein the metal nanostructure is a dendrimer type metal nanostructure and the metal nanostructure has a 2-dimensional or 1-dimensional structure wherein a plurality of branches are formed therein and nanogaps are present between the branches.

19. The metal nanostructure according to claim 17, wherein the dendrimer type or dendrimer-derived metal nanostructure has a 2-dimensional structure with horizontal and vertical sizes of 10 nm or more and 100 nm or less and a thickness of 1-10 nm, or the dendrimer type or dendrimer-derived metal nanostructure has a 1-dimensional structure with one of horizontal and vertical sizes of 10 nm or more and 100 nm or less and the other vertical or horizontal size and a thickness of 1-10 nm.

20. The metal nanostructure according to claim 18, wherein the metal nanostructure has a primary branch that has grown from a metal nanoparticle nucleus and n-th (n is an integer which is 2 or more) branches that have grown from the primary branch, and has nanogaps between the primary branch and the n-th branch, between the n-th branches, or between the primary branch and the n-th branch and between the n-th branches.

21. The metal nanostructure according to claim 20, wherein the metal nanostructure has horizontal and vertical sizes of 50-60 nm and a thickness of 4-5 nm.

22. The metal nanostructure according to claim 20, wherein a size of the nanogap is 2-8 nm.

23. The metal nanostructure according to claim 20, wherein a surface area of the metal nanostructure is 2-3 times that of a spherical particle of the same volume.

24. The metal nanostructure according to claim 17, wherein a metal of the metal nanostructure is one or more selected from a group consisting of Ag, Au, Cu, Pt, Fe, Co, Ni, Ru, Rh and Pd.
